(19) United States
(12) Patent Application Publication
(10) Pub. No.: US 2007/0270862 A1
(43) Pub. Date: Nov. 22, 2007

(54) Instruments and methods for preparing an intervertebral space

(75) Inventors: Kidong Yu, Memphis, TN (US); Danny Braddock JR., Germantown, TN (US); Eric Lange, Collierville, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/393,488

(22) Filed: Mar. 30, 2006

(51) Int. Cl. A61B 17/00

(52) U.S. Cl. 606/79

(57) ABSTRACT

A surgical instrument for use in spinal surgery is provided. The surgical instrument may have particular application in posterior total disc arthroplasty. The surgical instrument is adapted to be minimally invasive. The surgical instrument can be anchored into position by a stability pin. Further, the surgical instrument can guide a chisel or other instrument to the intervertebral space to resect a portion of a vertebral endplate. In at least one embodiment, the surgical instrument is also capable of distracting the disc space.

### TECHNICAL FIELD

Embodiments of the invention relate generally to devices and methods for accomplishing spinal surgery, and more particularly in some embodiments, to devices and methods of preparing the intervertebral disc space for spinal arthroplasty from a posterior approach. Various implementations of the invention are envisioned, including use in total spine arthroplasty for nerve retraction, disc restoration, and preparation of the intervertebral space using a minimally invasive procedure via a posterior approach.

### BACKGROUND

Often spinal implants are inserted via a posterior approach, including posterolateral approaches. Implantation via a posterior approach can cause damage to surrounding tissue and lead to other complications. In some embodiments, the implants described in the following prior patent applications, incorporated herein in their entirety by reference, are adapted for implantation via a posterior approach:
U.S. Utility patent application Ser. No. 11/031,602, filed on Jan. 7, 2005 and entitled “Spinal Arthroplasty Device and Method;”
U.S. Utility patent application Ser. No. 11/031,603, filed on Jan. 7, 2005 and entitled “Dual Articulating Spinal Device and Method;”
U.S. Utility patent application Ser. No. 11/031,780, filed on Jan. 7, 2005 and entitled “Split Spinal Device and Method;”
U.S. Utility patent application Ser. No. 11/031,904, filed on Jan. 7, 2005 and entitled “Interconnected Spinal Device and Method;”
U.S. Utility patent application Ser. No. 11/031,700, filed on Jan. 7, 2005 and entitled “Support Structure Device and Method;”
U.S. Utility patent application Ser. No. 11/031,783, filed on Jan. 7, 2005 and entitled “Mobile Bearing Spinal Device and Method;”
U.S. Utility patent application Ser. No. 11/031,781, filed on Jan. 7, 2005 and entitled “Centrally Articulating Spinal Device and Method;” and
U.S. Utility patent application Ser. No. 11/031,903, filed on Jan. 7, 2005 and entitled “Posterior Spinal Device and Method.”

Accordingly, there is a need for improved instrumentation and methods that avoid the drawbacks and disadvantages of the known methods, devices, and surgical techniques.

### SUMMARY

In one embodiment, a surgical instrument for providing a working area in a spinal surgery is provided.

In a second embodiment, a surgical instrument for use in spinal surgery is provided. The surgical instrument includes a body having a proximal end and a distal end with a longitudinal axis extending at least partially between the proximal end and the distal end. A distractor portion disposed adjacent the distal end and is adapted for distracting the intervertebral space of a superior vertebra and an inferior vertebra. A stabilizing device passage extends from the proximal end of the surgical instrument at least partially along the longitudinal axis and is adapted to receive a stabilizing device. A tool passage also extends from the proximal end of the surgical instrument at least partially along the longitudinal axis and is adapted to guide a surgical tool to the intervertebral space.

In a third embodiment, a method of preparing a superior vertebra and an inferior vertebra of an intervertebral space to receive a spinal implant is provided. The method includes providing a chisel guide and a stability pin, creating a first exposure through a patient's back to access the intervertebral space, positioning the chisel guide into the intervertebral space, anchoring the chisel guide in position with the stability pin; and inserting a chisel into the intervertebral space to resect a portion of at least one of the superior vertebra or the inferior vertebra.

Additional and alternative features, advantages, uses, and embodiments are set forth in or will be apparent from the following description, drawings, and claims.

### BRIEF DESCRIPTION OF THE DRAWINGS

FIG. 1 is a top perspective view of a surgical instrument according to one embodiment of the present invention.

FIG. 2 is a side view of the surgical instrument of FIG. 1.

FIG. 3 is a rear view of the surgical instrument of FIG. 1.

FIG. 4 is a cross-section side view of the surgical instrument of FIG. 1.

FIG. 5 is a bottom perspective view of the surgical instrument of FIG. 1.

FIG. 6 is a perspective view of the surgical instrument of FIG. 1 engaged with an inserter.

FIG. 7 is a lateral view of the surgical instrument of FIG. 1 disposed between a superior vertebra and an inferior vertebra.

FIG. 8 is a perspective view of a chisel according to one embodiment of the present invention.

FIG. 9 is a side view of the chisel of FIG. 8.

FIG. 10 is a lateral view of the chisel of FIG. 8 being placed within the surgical instrument of FIG. 1 that is secured to the inferior vertebra by a stability pin

FIG. 11 is a perspective view of a chisel according to another embodiment of the present invention.

FIG. 12 is a lateral view of the chisel of FIG. 11 being utilized in combination with surgical instrument of FIG. 1.

FIG. 13 is a top view of the chisel being utilized in combination with the surgical instrument of FIG. 12.

FIG. 14 is a perspective view of a chisel tube according to another embodiment of the present invention being placed into the intervertebral space between a superior vertebra and an inferior vertebra and used in combination with distractors and a guide wire.

FIG. 15 is a perspective view of the chisel tube of FIG. 14 secured to the inferior vertebra via a stability pin.

FIG. 16 is a perspective view a chisel used in combination with the chisel tube of FIG. 15.

### DESCRIPTION

The present disclosure relates generally to vertebral reconstructive devices, and more particularly, to devices and procedures for performing spinal arthroplasty. For the purposes of promoting an understanding of the principles of the invention, reference will now be made to the embodiments, or examples, illustrated in the drawings and specific language will be used to describe the embodiments. It will nevertheless be understood that no limitation of the scope of the invention is intended. Any alterations and further modifications of the described embodiments, and any further applications of the principles of the invention as described herein are contemplated as would normally occur to one skilled in the art to which the invention relates.

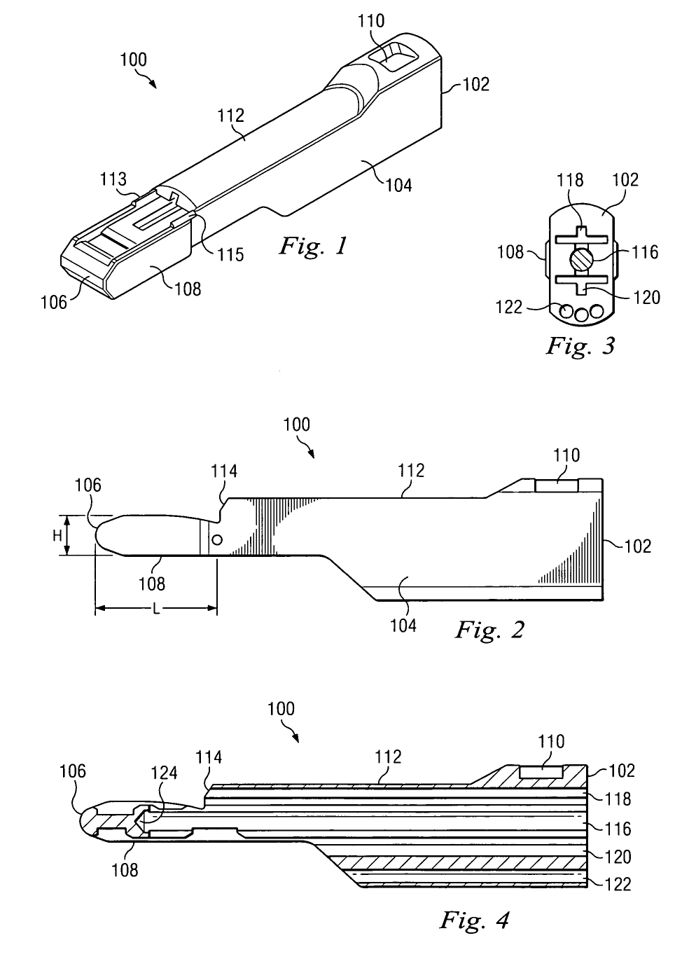

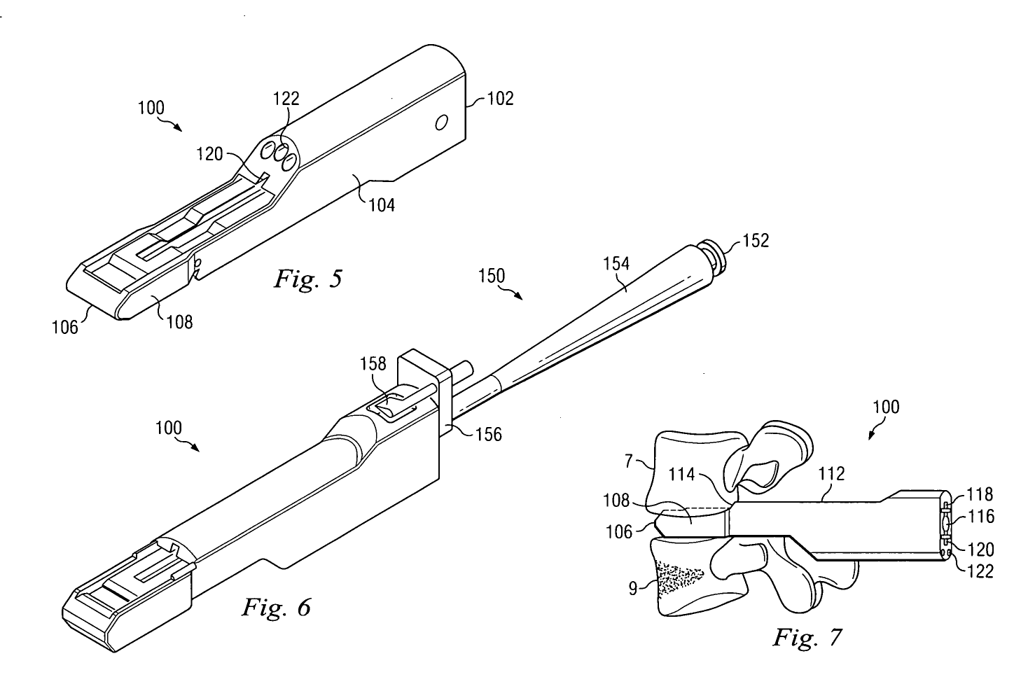

Referring now to FIGS. 1-5, shown therein is an exemplary embodiment of a surgical instrument 100 according to the present invention. The surgical instrument 100 includes a proximal end 102, a main body 104, and a distal end 106. The external features of the surgical instrument 100 include a distractor portion 108, an inserter recess 110, an upper surface 112, and an endplate engagement portion 114. The surgical instrument 100 also includes internal features, such as instrument passages 116, 118, 120, stability pin passages 122, and a stop portion 124.

Disposed adjacent the distal end 106 is a distractor portion 108. The distractor portion 108 is adapted to distract or restore the disc space between adjacent vertebrae. To this end, the distractor portion 108 may be tapered as it approaches the distal end 106 to facilitate engagement of a collapsed disc space. Further, the upper and lower edges or surfaces of the distractor portion 108 may be arcuate to match the shape of the superior and inferior vertebrae, respectively, once inserted into the disc space. The distractor portion 108 may be substantially similar to the Capstone Distractor from Medtronic, Inc.

As shown best in FIG. 2, the distractor portion 108 has a height H. The height H is configured to be of the appropriate size to restore the disc space of the patient. For this reason, it is fully contemplated that the height H may need to be different for different patients. Thus, it is contemplated that surgical instrument 100 may be part of a kit that includes a plurality of attachable distractor portions of various heights. In this regard, the distractor portions may be selectively engaged to the surgical instrument 100 so that the surgeon may choose the distractor portion with the appropriate height during surgery. Further, it is similarly contemplated that a length L of the distractor portion 108 may need to be different for different patients. Thus, the surgical instrument 100 may include a plurality of distractor portions having different lengths, different heights, or different heights and lengths from which the appropriate sized distractor portion may be selected. Further, as shown best in FIG. 3, the distractor portion 108 may have a width greater than the width of the main body 104 or proximal end 102 of the surgical instrument 100. However, in other embodiments the distractor portion 108 may have a width less than the width of the main body 104 or proximal end 102.

The upper surface 112 extends substantially along the length of the main body 104. The upper surface 112 is provided to protect the nerves, ligaments, and other soft tissue from instruments, such as chisels, used in combination with the surgical instrument 100. In other embodiments, the upper surface 112 may extend along only a portion of the main body 104. Towards the distal end of the upper surface 112 is the endplate engagement portion 114. As shown in FIG. 7, the endplate engagement portion 114 is adapted to engage the endplate of the superior vertebra once the distractor portion 108 has been positioned within the disc space. In this regard, the endplate engagement portion 114 may be shaped to mate with the contours of the endplate. As shown, the endplate engagement portion 114 may be comprised of a beveled edge of upper surface 112. The endplate engagement portion 114 provides additional stability to the surgical instrument 100 once inserted. In some embodiments, the endplate engagement portion 114 may also include protrusions 113, 115 (shown in FIGS. 1 and 6) adapted to engage the endplate to provide additional stability.

The surgical instrument 100 includes passages 116, 118, 120. The passages 116, 118, 120 will be described as separate passages for simplicity. In the present embodiment, the passages 116, 118, 120 may be different portions of a single passage or otherwise connected to one another. The passages 116, 118, 120 are adapted to receive other surgical instruments for use in spinal surgery, in particular instruments used to prepare the intervertebral space, as discussed below. In this respect, the intervertebral space is intended to include all areas that require preparation for spinal surgery, not just the disc space. Thus, the intervertebral space includes areas that are both intradiscal and extradiscal.

As shown in FIGS. 3 and 4, passage 116 has an opening at only the proximal end 102 of the surgical instrument 100 while passages 118, 120 are open at the proximal end and the opposing end. The opposing end of passage 116 is the stop portion 124 rather than a passage. The stop portion 124 serves to limit the travel of the surgical instruments used in combination with surgical instrument 100. For example, the stop portion 124 may serve to limit how far into the disc space a surgical instrument is able to travel. As with the distractor portion 108, the stop portion 124 may be sized or positioned appropriately to fit the characteristics of the patient. Further, as shown in FIG. 4, the stop portion 124 may be within the distractor portion 108 and, therefore, the dimensions of the stop portion may correlate with the appropriate size for the distractor portion. On the other hand, the surgical instrument may be sized to hit the stop portion at the appropriate position based on the anatomy of the patient.

Referring now to FIG. 6, shown therein is the surgical instrument 100 engaged with an inserter 150. The inserter 150 includes a proximal portion 152, a body 154, and an engagement end 156. The proximal portion 152 is adapted to receive a force from a driver, such as a mallet, hammer, slap-hammer, or similar device to encourage the surgical instrument 100 into the intervertebral space. The engagement end 156 is adapted for engaging the surgical instrument 100 to facilitate positioning of the surgical instrument into the intervertebral space. As shown, the engagement end 156 may include a projection or lever arm 158 adapted to selectively engage the inserter recess 110 of the surgical instrument. To this end, the lever arm 158 may be moveable so that it may be easily disengaged from the inserter recess 110 once the surgical instrument 100 is appropriately positioned. Though not expressly shown, it is fully contemplated that the inserter 150 may be shaped or otherwise include projections to engage passages 116, 118, 120 during insertion of the surgical instrument 100. However, in the present embodiment the inserter 150 does not interfere with access to passages 122. This is so a stability pin may be inserted while the inserter 150 is still engaged with the surgical instrument. This can be advantageous because the inserter 150 may be used to maintain the surgical instrument 100 at the proper approach until the pin properly secures the surgical instrument. Though the surgical instrument 100 has been described only as being inserted by inserter 150, it is fully contemplated that any device capable of positioning the surgical instrument may be utilized.

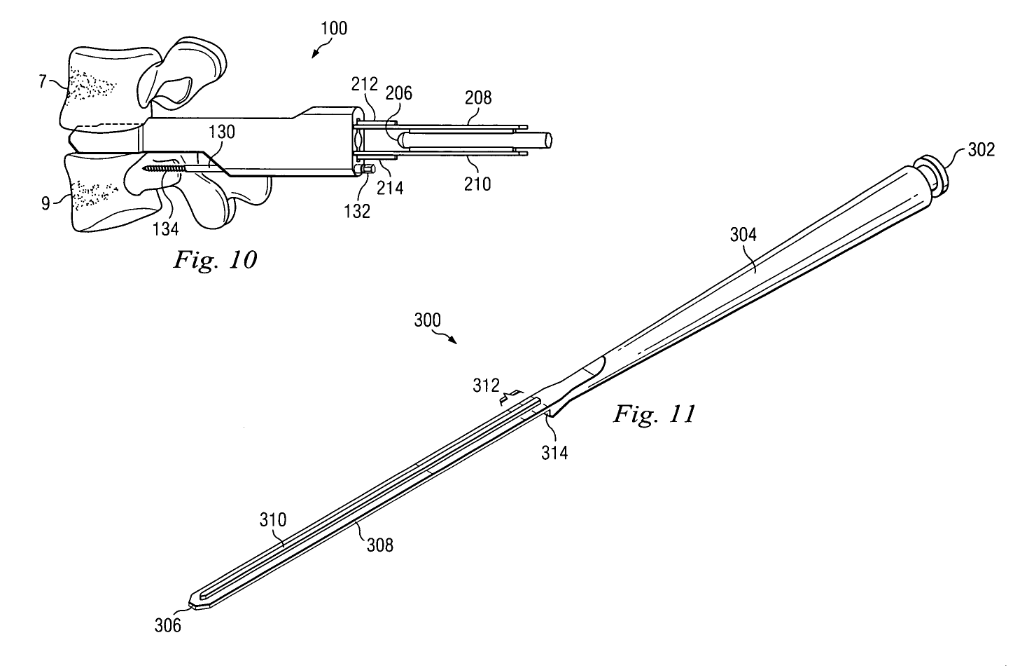

Referring now to FIG. 7, shown therein is the surgical instrument 100 inserted into the disc space between a superior vertebra 7 and an inferior vertebra 9. In the present embodiment, when properly positioned from a posterior approach, the distal end 106 of the surgical instrument 100 does not extend beyond the anterior portions of the vertebrae 7, 9. Further, the endplate engagement portion 114 engages a posterior portion of the superior vertebra's endplate. In practice, when the surgical instrument 100 is properly positioned a stability pin (FIG. 10) is inserted through a passage 122 and secured to the pedicle of the lower vertebra 9, thereby securing the surgical instrument at the proper approach. While the stability pin is described as being secured to the pedicle, it is fully contemplated that the pin may attach to any other convenient point to secure to the positioning of the surgical instrument 100.

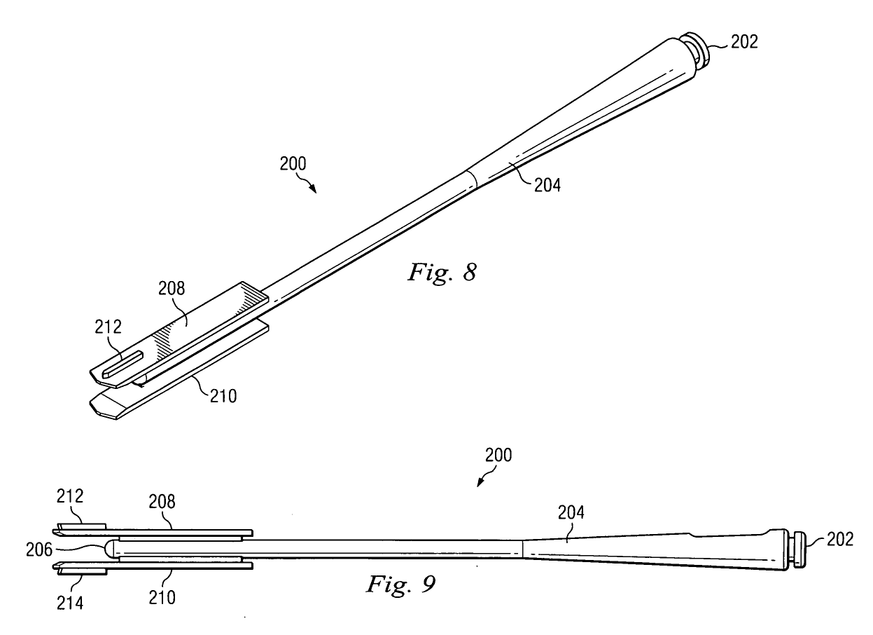

Referring to FIGS. 8 and 9, shown therein is a chisel 200 adapted for preparing the intervertebral space of adjacent vertebrae 7, 9. The chisel 200 includes a proximal portion 202, a body 204, and two cutting portions 208, 210. The proximal portion 202 is adapted to receive a force from a mallet, hammer, slap-hammer or similar device which is transferred to cutting portions 208, 210 to prepare the intervertebral space. Cutting portions 208, 210 are attached to the body 204 near a distal end 206 of the body. As shown, the cutting portions 208, 210 extend distally beyond the distal end 206 of the body 204. The cutting portions 208, 210 are shaped to substantially match the type of implant being inserted. For example, cutting portions 208, 210 include keel cutters 212, 214, respectively. The keel cutters 212, 214 are adapted to prepare a recess in the intervertebral space adapted to receive a keel of an implant. Keels are often used in spinal implants. For example, the Maverick spinal implant from Medtronic (see e.g., U.S. patent application Ser. No. 10/773,494 filed Feb. 12, 2004, herein incorporated by reference in its entirety) includes a keel. U.S. Patent Application Publication No. 2005/0171610 A1, herein incorporated by reference in its entirety, also discloses a spinal implant that includes a keel. Cutting portion 208 and cutting portion 210 need not be the same. For example, it is contemplated that the upper portion of the implant may include a keel while the lower portion does not. In that case, the cutting portion 208 would include a keel cutter, while cutting portion 210 would not. Any number of combinations may be utilized depending on the characteristics of the implant, or multiple implants, to be used.

As previously described, the passages 116, 118, 120 are adapted to receive other surgical instruments, such as chisel 200. In this embodiment, passage 116 is adapted to receive a body portion 204 and passages 118, 120 are adapted to receive cutting portions 208, 210 of the chisel 200. In particular, the passages 118, 120 are adapted to receive the keel cutters 212, 214 of cutting portions 208, 210, respectively. Further, the passages 118, 120 are adapted to guide the cutting portions 208, 210 of the chisel 200 to the intervertebral space so that the cutting portions are positioned properly to prepare the intervertebral space. As previously described, the stop portion 124 at the opposing end of passage 116 may be used to limit the travel of the chisel 200 into the disc space. In this case, the stop portion 124 is adapted to engage the distal end 206 of the body 204 of chisel 200 when the chisel reaches the appropriate distance into the disc space.

As seen in FIG. 10, surgical instrument 100 may be secured in the appropriate position by stability pin 130. Stability pin 130 includes a proximal portion 132 adapted to be engaged by a driver. The driver may facilitate insertion of the stability pin 130 by rotation, similar to a screw driver or torque driver, or direct force, similar to a hammer or slap-hammer. In the present embodiment, stability pin 130 also includes engagement portion 134. Engagement portion 134 is adapted to engage the pedicle of the inferior vertebra 9 and may be threaded or otherwise similar to a bone screw or other fixation device. In this regard, it is contemplated that the pedicle may be pre-drilled to receive the stability pin 130. Further, engagement portion 134 may be adapted to engage any other convenient portion of the vertebra or adjacent structures.

Once the surgical instrument 100 has been secured in the appropriate position by stability pin 130, the passage 118 opens substantially at the endplate of the superior vertebra 7. Passage 120, on the other hand, is configured to open prior to reaching a pedicle of the lower vertebra 9. Thus, as chisel 200 is inserted into the passages 116, 118, 120 of surgical instrument 100 the cutting portions 208, 210 will be guided to the appropriate positions and at the appropriate angle to prepare the intervertebral space. With passage 118 abutting the end plate of superior vertebra 7, cutting portion 208 and its keel cutter 212 will chisel or cut away a passage in the superior vertebra. With passage 120 opening before a pedicle of inferior vertebra 9, cutting portion 210 and its keel cutter 214 can shape the parts of the vertebra, including portions of the pedicle or remaining facet in some embodiments, needed to prepare the intervertebral space.

The surgical instrument 100 is adapted for use on one side of the intervertebral space. Thus, by utilizing a pair of surgical instruments 100 both sides of the intervertebral space can be prepared for a bilateral procedure. In some instances this may be advantageous because as one side of the intervertebral space is distracted, the other side may collapse. However, in one embodiment each side of the intervertebral space may be prepared separately using a single surgical instrument 100. On the other hand, by using a chisel 200 with two cutting portions 208, 210 both the superior and inferior portions of the each side of the intervertebral space can be prepared simultaneously. Again, in other embodiments the superior and inferior portions of the intervertebral space may be prepared separately.

FIG. 11 shows a chisel 300 adapted to prepare the superior and inferior portions of the intervertebral space separately. Chisel 300 includes a proximal portion 302, a body 304, and a distal end 306. Chisel 300 may be substantially similar to chisel 200 described above. For this reason, only certain features of chisel 300 will be described herein. A cutting portion 308 is disposed adjacent the distal end 306. As with chisel 200, the cutting portion 308 is adapted for preparing the intervertebral space by removing unwanted bone or other tissue and may include a keel cutter 310.

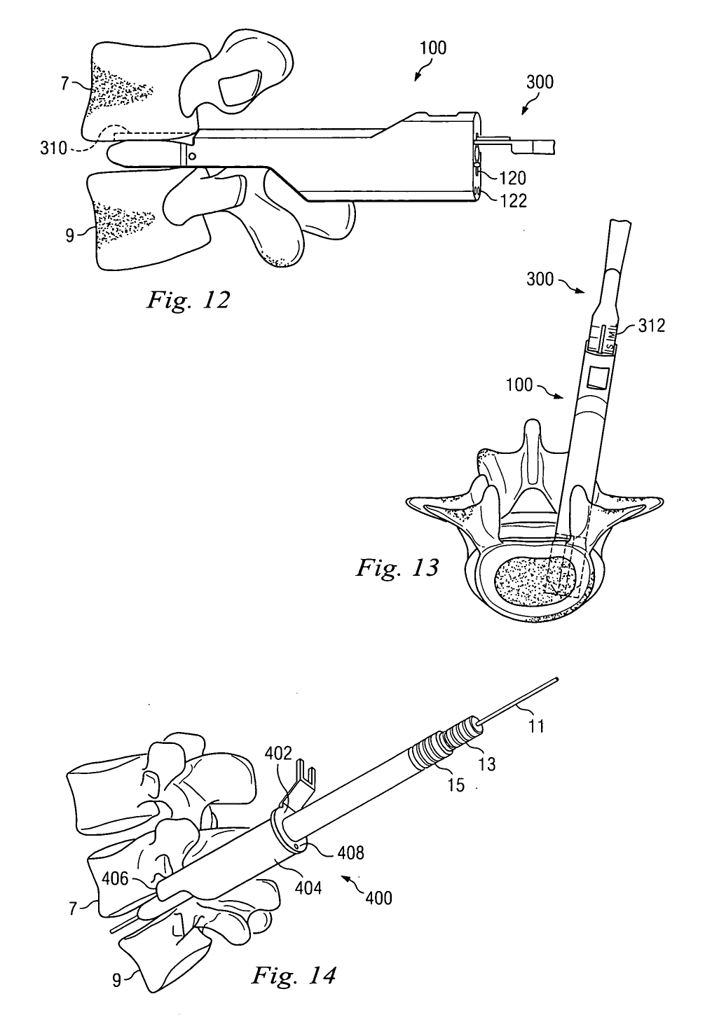

As shown in FIG. 12, chisel 300 may be inserted through passage 118 to prepare the superior portion of the intervertebral space then removed and inserted through passage 120 to prepare the inferior portion of the intervertebral space, or vice-versa. In this regard, the chisel 300 include markers 312 that indicate to the surgeon the relative distance the cutting portion 308 has traveled into the disc space. The surgeon may determine how far to insert the chisel 300 based upon the patient's anatomy, the implant to be inserted, a combination of the two, or any other characteristic the surgeon deems appropriate. Further, stop portion 314 may be utilized to limit the distance the chisel 300 is able to travel into the disc space. The stop portion 314 is shaped so that it is unable to pass into the passages 116, 118, 120 of the chisel 100. Thus, stop portion 314 may be positioned such that it abuts the proximal end 102 of the surgical instrument 100 to limit the distance the cutting portion 308 can travel into the disc space. In this regard, the markers 312 and the stop portion 314 may be used in combination or individually to ensure proper preparation.

Though not shown, it is fully contemplated that the cutting portion 308 may be shaped or otherwise include an additional member that extends substantially along the length of the cutting portion 308 such that when the chisel 300 is inserted into surgical instrument 100, the additional member will hit the stop portion 124 when chisel 300 is fully inserted. In one embodiment the additional member or shape of the cutting portion 308 need not extend substantially along the length of the cutting portion, but rather may be a single protrusion positioned to abut the stop portion 124 when fully inserted. This additional feature may be in addition to or in replace of the markers 312 and stop portion 314 features described above.

Surgical instrument 100 may be utilized in various surgical procedures related to the spine. Surgical instrument 100 has particular application for total disc arthroplasty. Further, the surgical instrument 100 can be utilized in a minimally invasive technique for total disc arthroplasty. An example of a surgical technique utilizing the surgical instrument 100 in a total disc arthroplasty will now be described. It is fully understood, however, that variations to the procedure are contemplated as one with skill in the art would recognize without leaving the scope of the present invention. Further, though the surgical instrument 100 can be used in a bilateral procedure, the procedure below will be described with respect to only a single side of the intervertebral space. It being understood that the procedure may be substantially similar for the opposing side.

First, an exposure is created through the patient's back to gain access to the intervertebral space. After gaining access to the intervertebral space, portions of the facet joint are removed. Removing portions of the facet joint provides the surgical instrument 100 with a clearer path to the disc space. However, before inserting the surgical instrument 100 the nerves must be retracted to allow the surgical instrument to pass by without causing damage. Once the nerves are retracted the surgical instrument 100 may then be inserted and driven into the intervertebral space by inserter 150. Where multiple sized distractor portions 108 are available, the surgeon will start with the smallest size. If the first distractor portion fits loosely, then the surgeon will try the next higher size until the proper fit is found. Once the distractor portion 108 is sized properly the surgical instrument 100 must be positioned properly. The proper trajectory for the surgical instrument 100 may be determined by fluoroscopy.

Once the surgical instrument 100 is positioned correctly the stability pin 130 may be inserted to anchor the surgical instrument in place. After the surgical instrument 100 is positioned properly and secured by the stability pin 130, a chisel may be inserted through the surgical instrument to prepare the intervertebral space. The surgical instrument 100 serves to guide the chisel along the proper trajectory to the intervertebral space while protecting the surrounding tissue. By tapping the chisel through the tube and into the intervertebral space the endplates of the adjacent vertebrae 7, 9 may be prepared. Further, utilizing a chisel similar to chisels 200, 300 described above, the intervertebral space may carved straight and flat, but with a keel receiving recess, in preparation for receiving an implant. This is not necessary however. After the chiseling is complete, the stability pin 130 and the surgical instrument 100 may be removed. Then the implant may be inserted into the prepared intervertebral space.

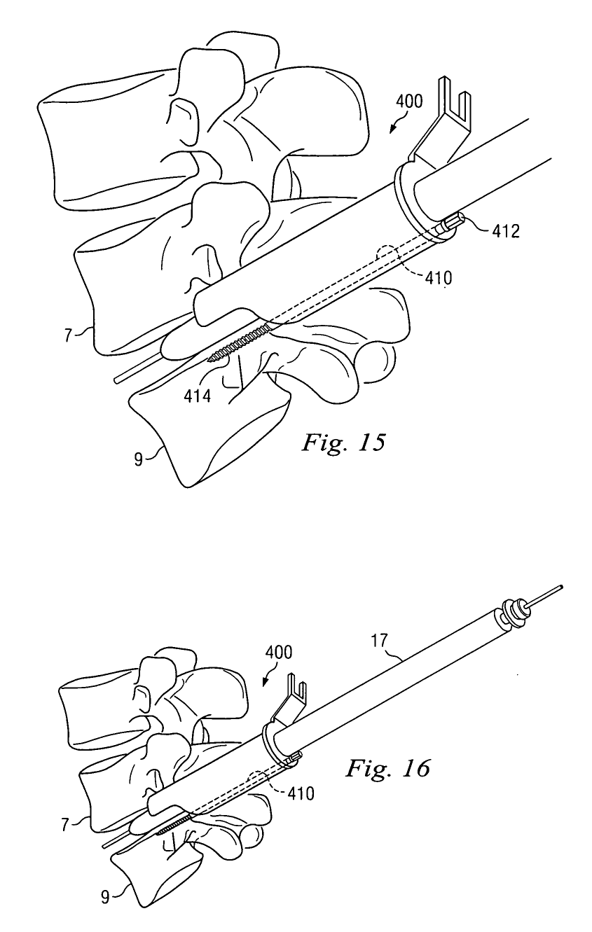

Referring now to FIGS. 14-16, shown therein is another exemplary embodiment of a surgical instrument 400 according to the present invention. Surgical instrument 400 may be substantially similar to surgical instrument 100 described and, therefore, only certain aspects will be described. Surgical instrument 400 includes a proximal end 402, a main body 404, and a distal end 406. The surgical instrument 400 may be shaped to limit its invasiveness. For example, the surgical instrument 400 may be substantially flat or otherwise contoured on the side disposed towards the centerline of the body to avoid the spinous processes. In that respect, the surgical instrument 400 may be a pair surgical instruments each adapted for placement on either the right side or the left side for use in bilateral procedure.

The surgical instrument also includes two passages, one passage to create a workspace for additional surgical instruments and a passage 408 adapted to receive a stability pin 410. Stability pin 410 may be substantially similar to stability pin 130 described above. Stability pin 410 includes a proximal portion 412 adapted to be engaged by a driver. The driver may facilitate insertion of the stability pin 410 by rotation or direct force. Stability pin 410 also includes engagement portion 414. Engagement portion 414 is adapted to engage the pedicle of the inferior vertebra 9 and may be threaded or otherwise similar to a bone screw or other fixation device. However, engagement portion 414 may be adapted to engage any other convenient portion of the vertebra or adjacent structures.

Surgical instrument 400 is different from surgical instrument 100 in that it is not adapted to distract the disc space. Rather, dilators 13, 15 may be utilized in combination with a guide wire 11 to distract the disc space, as shown in FIG. 14. While retracting the nerves, the surgical instrument 400 may be positioned over the dilators 13, 15 and adjacent the posterior portion of the vertebral body. Fluoroscopy may be utilized to determine the best trajectory for the surgical instrument 400. Again once the best trajectory is determined and the surgical instrument 400 is aligned, the stability pin 410 may be inserted to secure the surgical instrument into place. At that point, the dilators 13, 15 may be removed as the surgical instrument 400 may support the disc space. Then chisel 17 may be inserted along the guide wire 11 through the passage in the surgical instrument 400 to prepare the intervertebral space. After the intervertebral space is prepared, the stability pin 410 and the surgical instrument 400 may be removed and the implant inserted into the prepared intervertebral space.

The surgical instruments, 100, 200, 300, 400 described above may be made of materials suitable for surgical procedures. For example, it is fully contemplated that metals, such as stainless steel and titanium, and polymers, such as polyetheretherketone (PEEK), may be used.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” and “right,” are for illustrative purposes only and can be varied within the scope of the disclosure. In the claims, means-plus-function clauses are intended to cover the structures described herein as performing the recited function and not only structural equivalents, but also equivalent structures.

What is claimed is:

1. A surgical instrument for use in spinal surgery, comprising:
a body having a proximal end, a distal end, and a longitudinal axis extending at least partially between the proximal end and the distal end;
a distractor portion disposed adjacent the distal end and adapted for distracting an intervertebral space between a superior vertebra and an inferior vertebra;
a stabilizing device passage extending from the proximal end at least partially along the longitudinal axis and adapted to receive a stabilizing device; and
a tool passage extending from the proximal end at least partially along the longitudinal axis and adapted to guide a surgical tool to the intervertebral space.

2. The surgical instrument of claim 1, wherein the stabilizing device passage is further adapted to guide the stabilizing device towards a pedicle of the inferior vertebra.

3. The surgical instrument of claim 2, wherein the stabilizing device is a pin adapted to engage the pedicle of the inferior vertebra.

4. The surgical instrument of claim 3, wherein the pin is at least partially threaded.

5. The surgical instrument of claim 1, wherein the surgical tool is a chisel.

6. The surgical instrument of claim 5, wherein the chisel includes an upper portion configured to prepare the superior vertebra and a lower portion configured to prepare the inferior vertebra.

7. The surgical instrument of claim 6, wherein at least one of the upper portion or the lower portion to includes a keel cutter.

8. The surgical instrument of claim 7, wherein the tool passage includes a superior portion adapted to guide the upper portion of the chisel at least partially towards the superior vertebra and an inferior portion adapted to guide the lower portion of the chisel at least partially towards the inferior vertebra.

9. The surgical instrument of claim 5, wherein the tool passage is further adapted to protect a surrounding tissue.

10. The surgical instrument of claim 9, wherein the surrounding tissue includes a nerve.

11. The surgical instrument of claim 1, further comprising a stop portion adapted to limit the distance the surgical instrument can travel into the intervertebral space.

12. The surgical instrument of claim 11, wherein the stop portion is located within the distractor portion.

13. The surgical instrument of claim 11, wherein the stop portion is positioned to match a size of the intervertebral space.

14. The surgical instrument of claim 1, wherein the distractor portion is sized to match a size of the intervertebral space.

15. The surgical instrument of claim 1, wherein the tool passage includes a superior portion adapted to the surgical tool at least partially towards the superior vertebra and an inferior portion adapted to guide the surgical tool at least partially towards the inferior vertebra.

16. A chisel guide for use in disc arthroplasty, comprising:
a body having a proximal end, a distal end, and a longitudinal axis extending at least partially between the proximal end and the distal end;
a stabilizing device passage extending from the proximal end at least partially along the longitudinal axis and adapted to receive a stabilizing device; and a
a tool passage extending from the proximal end at least partially along the longitudinal axis and adapted to provide access to a intervertebral space between a superior vertebra and an inferior vertebra for a chisel.

17. The chisel guide of claim 16, wherein the body is substantially cylindrical.

18. The chisel guide of claim 16, wherein the body is shaped to avoid interfering with a spinous process upon insertion.

19. The chisel guide of claim 18, wherein the body includes a substantially flat side extending at least partially along the longitudinal axis.

20. The chisel guide of claim 16, wherein the stabilizing device passage is further adapted to guide the stabilizing device towards a pedicle of the inferior vertebra.

21. The chisel guide of claim 20, wherein the stabilizing device is a pin adapted to engage the pedicle of the inferior vertebra.

22. A method of preparing a superior vertebra and an inferior vertebra of an intervertebral space to receive a spinal implant, comprising:
providing a first chisel guide and a first stability pin, the first chisel guide including a first stability pin passage adapted to receive the first stability pin and a first tool passage adapted to guide a chisel towards the intervertebral space, the first stability pin adapted to anchor the first chisel guide to a portion of the inferior vertebra;
creating a first exposure through a patient's back to access the intervertebral space;
positioning the first chisel guide into the intervertebral space;
anchoring the first chisel guide in position by inserting the first stability pin; and
inserting a chisel into the intervertebral space through the first tool passage of the first chisel guide to resect a portion of at least one of the superior vertebra or the inferior vertebra.

23. The method of claim 22, wherein the first stability pin is adapted to anchor the first chisel guide to a pedicle of the inferior vertebra.

24. The method of claim 23, wherein anchoring the first chisel guide in position includes engaging the first stability pin to the pedicle of the inferior vertebra.

25. The method of claim 24, wherein the first stability pin is at least partially threaded.

26. The method of claim 22, wherein a portion of both the superior vertebra and inferior vertebra are resected.

27. The method of claim 26, wherein the portion of the superior vertebra is resected separately from the portion of the inferior vertebra.

28. The method of claim 26, wherein the portion of the superior vertebra and the portion of the inferior vertebra are resected simultaneously.

29. The method of claim 28, wherein resecting a portion of the superior vertebra includes creating a recess to receive a keel of the spinal implant.

30. The method of claim 29, wherein resecting a portion of the inferior vertebra includes creating a recess to receive a keel of the spinal implant.

31. The method of claim 22, further comprising resecting at least a portion of at least one natural facet.

32. The method of claim 22, further comprising:
providing a second chisel guide and a second stability pin, the second chisel guide including a second stability pin passage adapted to receive the second stability pin and a second tool passage adapted to guide a chisel towards the intervertebral space, the second stability pin adapted to anchor the second chisel guide to a portion of the inferior vertebra;
creating a second exposure through a patient's back to access the intervertebral space;
positioning the second chisel guide into the intervertebral space;
anchoring the second chisel guide in position by inserting the second stability pin; and
inserting a chisel into the intervertebral space through the second tool passage of the second chisel guide to resect a portion of at least one of the superior vertebra or the inferior vertebra.

33. The method of claim, wherein the second exposure is on an opposite side of the patient's vertebral canal from the first exposure.

34. A kit for use in spinal surgery, comprising:
a surgical instrument having a stabilizing device passage adapted to receive a stabilizing device, a tool passage adapted to guide a chisel to an intervertebral space between a superior vertebra and an inferior vertebra, and an attachment portion adapted to selectively engage a distractor portion;
a first distractor portion configured to be selectively engaged by the attachment portion of the surgical instrument, adapted to distract the intervertebral space between the superior vertebra and the inferior vertebra, and having a first height;
a second distractor portion configured to be selectively engaged by the attachment portion of the surgical instrument, adapted to distract the intervertebral space between the superior vertebra and the inferior vertebra, and having a second height, the second height being greater than the first height; and
a stabilizing device.

35. The kit of claim 34, further comprising a third distractor portion configured to be selectively engaged by the attachment portion of the surgical instrument, adapted to distract the intervertebral space between the superior vertebra and the inferior vertebra, and having a third height, the third height being greater than the second height.

36. The kit of claim 34, wherein the first distractor portion has a first length and the second distractor portion has a second length, the second length being greater than the first length.

37. The kit of claim 34, further comprising a first chisel having a first cutting portion, the first cutting portion having a first cutting length.

38. The kit of claim 37, further comprising a second chisel having a second cutting portion, the second cutting portion having a second cutting length greater than the first cutting length.

39. The kit of claim 38, further comprising a third chisel having a third cutting portion, the third cutting portion having a third cutting length greater than the second cutting length.

40. The kit of claim 39, wherein the first cutting length substantially matches a length of a first distractor portion, the second cutting length substantially matches a length of a second distractor portion, and the third cutting length substantially matches a length of a third distractor portion.
